(19) United States
(12) Patent Application Publication
(10) Pub. No.: US 2010/0095958 A1
(43) Pub. Date: Apr. 22, 2010

(54) Pre-filled, single-use, disposable small volume medication nebulizer

(76) Inventors: Russell W. King, Sierra Madre, CA (US); Kenneth Michael McPeck, El Monte, CA (US)

Correspondence Address: JAMES E. BRUNTON, ESQ., P. O. BOX 29000, GLENDALE, CA, 91209, US

(21) Appl. No.: 12/587,486

(22) Filed: Oct. 6, 2009

Related U.S. Application Data
(63) Continuation-in-part of application No. 11/894,860, filed on Aug. 21, 2007.

(51) Int. Cl. A61M 11/00

(52) U.S. Cl. 128/200.18

(57) ABSTRACT

A miniaturized pre-filled, single-use, disposable, small volume medication nebulizer unit for medicinal use that delivers a mist of properly sized aerosol particles of medicament to the patient with a very high level of efficiency. The nebulizer can be effectively used in conjunction with conventional tee and mouthpiece patient interface devices as well as with more sophisticated interface devices such as dosimetric/reservoir systems, or mechanical ventilator systems.

### CROSS-REFERENCE TO RELATED APPLICATIONS

This is a Continuation-In-Part of U.S. Ser. No. 11/894,860 filed Aug. 21, 2007.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

The present invention relates generally to inhalation devices. More particularly, the invention concerns a miniaturized pre-filled, single-use, disposable, small volume nebulizer for medicinal use that delivers a mist of properly sized aerosol particles of medicament to the patient with a very high-level of efficiency.

### DESCRIPTION OF RELATED ART INCLUDING INFORMATION DISCLOSED UNDER 37 CFR 1.97 AND 1.98

In medicine, a nebulizer is defined as a device that is used to administer medication to the patient's airways in the form of a liquid mist, more properly known as an aerosol. In general the prior art devices used for producing medical aerosols fall into two categories; the small volume nebulizer (SVN), and the metered dose inhaler (MDI). The small volume nebulizer (SVN) has traditionally been the apparatus of choice for delivery of therapeutic aerosols. The delivery apparatus typically consists of a multi-use disposable or reusable nebulizer, a mouthpiece or facemask, and a pressurized gas source usually oxygen or air. The metered dose inhaler (MDI), on the other hand, typically contains the active drug, dissolved in chlorofluorcarbon (CFC) or chlorofluroalkane (CFA) propellants and excipients plus a metering valve. The drug-containing canister of the device is generally fitted to a mouthpiece actuator and spacer or valved holding chamber, and activation of the device by compressing it results in the release of a metered dose of medication.

Various types of prior art inhalers have also been offered for sale and are in wide use. Inhalers have the advantage of portability but have been criticized on the basis that patients often lack the coordination and psychomotor skills to use them properly without professional supervision. This dichotomy of available device types (nebulizers vs. inhalers) has lead to a great deal of controversy regarding which method is superior, although many experts have concluded that nebulizers and inhalers are essentially equivalent in terms of therapeutic outcomes. Accordingly, in many respects, the choice of device revolves around non-outcome related factors such as cost, convenience, ease-of-use, safety, patient preference, patient compliance and adherence, as well as the availability of medications in one or both delivery forms. Despite alternative methodologies, it is clear that inhaled medication delivery by nebulizer is a permanent part of the treatment options for respiratory disease patients and is becoming a useful tool for systemic drug delivery as well.

This being the case, there is an abundance of plastic disposable medication nebulizers on the market, but the vast majority of these devices are essentially clones, differing from one another mainly in appearance. Functionally, they are essentially identical. The overriding similarity between all these devices is that they are all supplied empty and the medication they are to nebulize must be transferred into them prior to commencement of the treatment by either the professional respiratory therapist in the hospital setting or the patient or patient's caregiver in the home setting.

Recently, various investigators and companies have sought to improve liquid nebulization. However, due to the physics of jet nebulization, the possibility of performance improvements in the jet nebulizer itself are very limited. Many of the improvements have involved electronic controlled or driven nebulizers that have improved efficiency but are also so expensive as to be out-of-reach for the typical routine nebulization purposes.

As will be discussed more fully hereinafter, there are various well recognized technical limitations to nebulizer use. These include the following:

1. Excessive patient dosing time.
2. Dose of drug delivered to the patient is undesirably affected by patient breathing parameters that may result in unacceptable variations in drug delivery dose.
3. Cleaning of the nebulizer after each use is time-consuming and frequently neglected thereby providing a possible avenue for nosocomial infection (bacteria/viral spread within a healthcare organization).
4. In a hospital environment, excessive amounts of technologist time is required for each patient treatment.
5. Release of the drug to atmosphere is not only wasteful but can be detrimental to healthcare workers who breathe “second-hand” aerosol drugs.
6. Because of lengthy treatment times, patients may become fatigued and compliance is compromised.
7. Hospital use is often determined by price only, not performance.

In light of the aforementioned drawbacks, Dr. J. H. Dennis, a highly recognized aerosol researcher, has stated as follows in the Practical Handbook of Nebulizer Therapy. London, Martin Dunitz; 2004: 42-43:
“It is clear that neither pressurized metered dose MDI's, nor DPI's meet all the necessary requirements despite the enormous amounts of pharmaceutical funding which has been devoted to improvement of these devices over the past three decades.”

It is this problem that the present invention seeks to address by providing the novel miniaturized pre-filled, single-use, disposable, small volume medication nebulizer unit of the invention.

### BRIEF SUMMARY OF THE INVENTION

It is an object of the present invention to provide a novel miniaturized pre-filled, single-use, disposable, small volume medication nebulizer unit for medicinal use that delivers a mist of properly sized aerosol particles of medicament to the patient with a very high-level of efficiency.

Another object of the invention is to provide a nebulizer of the aforementioned character that comprises a unique means for packaging an inhalation drug in a preferred unit-dose, single-use disposable container that confers the benefits of unit-dose packaging while it simultaneously performs the function of highly effective drug aerosolization.

Another object of the invention is to provide a nebulizer of the character described that is small in physical size for convenience of packaging, storage, dispensing and operation.

Another object of the invention is to provide a miniaturized, pre-filled, single-use, disposable, nebulizer that can be produced in large quantities at minimal cost by conventional thermoplastic injection molding means.

Another object of the invention is to provide a miniaturized nebulizer as described in the preceding paragraphs that can be effectively used in conjunction with conventional tee and mouthpiece patient interface devices as well as with more sophisticated patient interface devices such as dosimetric/reservoir systems, or mechanical ventilator systems.

Another object of the invention is to provide a miniaturized, pre-filled, single-use, disposable nebulizer that effectively mitigates against the possibility of self-contamination or cross-contamination due to improper cleaning of the device.

Another object of the invention is to provide a miniaturized nebulizer of the class described that effectively minimizes practitioner set-up and preparation time thereby conferring significant labor savings benefits upon healthcare organizations that employ such practitioners for the purpose of administering medicated aerosol therapy.

Another object of the invention is to provide a miniaturized, pre-filled, single-use, disposable nebulizer that effectively reduces or eliminates practitioner clean-up time following administration of the contained medication thereby conferring significant labor savings benefits upon healthcare organizations that employ such practitioners for the purpose of administering medicated aerosol therapy.

Another object of the invention is to provide a nebulizer of the type described in the preceding paragraphs that incorporates a miniature nebulizer attached to a dosimetric reservoir configuration that delivers superior patient dose consistency and repeatability even over a wide range of patient breathing parameters. This feature uniquely provides the ability to accurately predict the actual dose delivered to the patient. Such calculations may be made to within ±20% of that predicted when using the dose quantification equation discussed hereinafter.

Another object of the invention is to provide a miniaturized, pre-filled, single-use, disposable, nebulizer unit that, when combined with an appropriate dosimetric reservoir system, provides a substantial reduction of drug release to the ambient atmosphere thereby protecting caregivers and other personnel by reducing or minimizing exposure to “second-hand” aerosol drugs.

It is an object of the present invention to provide an inhalation apparatus which will deliver an aerosolized medication to the patient which comprises up to 70% of the medication aerosolized.

Another object of the invention is to provide the best features of both breath-enhancement and dosimetric/reservoir technology.

Yet another object of the apparatus is to provide for patient drug dose delivery rates of up to 5 times faster than that of typical nebulizer/tee configurations.

Another object of the invention is to provide delivered dose consistency even over a wide range of patient breathing parameters.

Another object of the invention is to provide an apparatus which will deliver to the patient essentially the same particle size distribution of the aerosol mist that originates from the nebulizer itself.

Yet another object of the invention is to provide means for safely filtering air exhaled from the patient before its release to room atmosphere.

### BRIEF DESCRIPTION OF SEVERAL VIEWS OF THE DRAWINGS

FIG. 1 is a generally perspective view of one form of the single dose disposable nebulizer unit of the invention with both closures in place as if filled with medication.

FIG. 2 is an enlarged, cross-sectional view taken along lines 2-2 of FIG. 1.

FIG. 3 is an exploded, generally perspective, cross-sectional view of the nebulizer unit illustrated in FIG. 2 of the drawings.

FIG. 4 is a generally perspective view illustrating the nebulizer unit of the invention interconnected with a patient delivery device.

FIG. 5 is a greatly enlarged, cross-sectional view taken along lines 5-5 of FIG. 4.

FIG. 6 is a view taken along lines 6-6 of FIG. 5.

FIG. 7 is a cross-sectional view taken along lines 7-7 of FIG. 5.

FIG. 8 is an enlarged, cross-sectional view taken along lines 8-8 of FIG. 5.

FIG. 9 is an enlarged cross-sectional view taken along lines 9-9 of FIG. 5.

FIG. 10 is a cross-sectional view taken along lines 10-10 of FIG. 5.

FIG. 11 is a fragmentary cross-sectional view of the upper portion of the nebulizer unit better illustrating the construction of the nebulizer assembly.

FIG. 12 is a generally perspective view, partly in cross section, showing one form of the nebulizer unit of the invention interconnected with a conventional mouthpiece, tee and aerosol flex tube reservoir.

FIG. 13 is an enlarged, cross-sectional view taken along lines 13-13 of FIG. 12.

FIG. 14 is a cross-sectional view taken along lines 14-14 of FIG. 13.

FIG. 15 is a cross-sectional view taken along lines 15-15 of FIG. 14.

FIG. 16 is a generally perspective view of an alternate form of the single dose disposable nebulizer unit of the present invention.

FIG. 17 is an exploded, generally perspective view of the nebulizer unit illustrated in FIG. 16 of the drawings.

FIG. 18 is an enlarged, generally perspective view of one form of the airflow baffle component of this latest form of the invention.

FIG. 19 is a cross-sectional view taken along lines 19-19 of FIG. 16.

FIG. 20 is a greatly enlarged, cross-sectional view taken along lines 20-20 of FIG. 19.

FIG. 21 is a greatly enlarged, cross-sectional view taken along lines 21-21 of FIG. 19.

FIG. 22 is a greatly enlarged, generally perspective fragmentary view of the upper right hand portion of FIG. 23.

FIG. 23 is a generally perspective view of still another form of the single dose disposable nebulizer unit of the present invention.

FIG. 24 is a partly broken away, generally perspective view of one form of the check valve diaphragm of the invention illustrated in FIG. 23.

FIG. 25 is a side elevation will view of the check valve diaphragm of the invention illustrated in FIG. 23

FIG. 26 is a cross-sectional view taken along lines 26-26 of FIG. 23.

FIG. 27 is an enlarged cross-sectional view taken along lines 27-27 of FIG. 26.

FIG. 28 is an enlarged view taken along lines 28-28 of FIG. 23.

FIG. 29 is an enlarged cross-sectional view taken along lines 29-29 of FIG. 23.

FIG. 30 is a greatly enlarged cross-sectional view illustrating the operation of the check valve of the apparatus of this latest form of the invention that controls fluid flow through the exit path from the third chamber of the device.

### DETAILED DESCRIPTION OF THE INVENTION

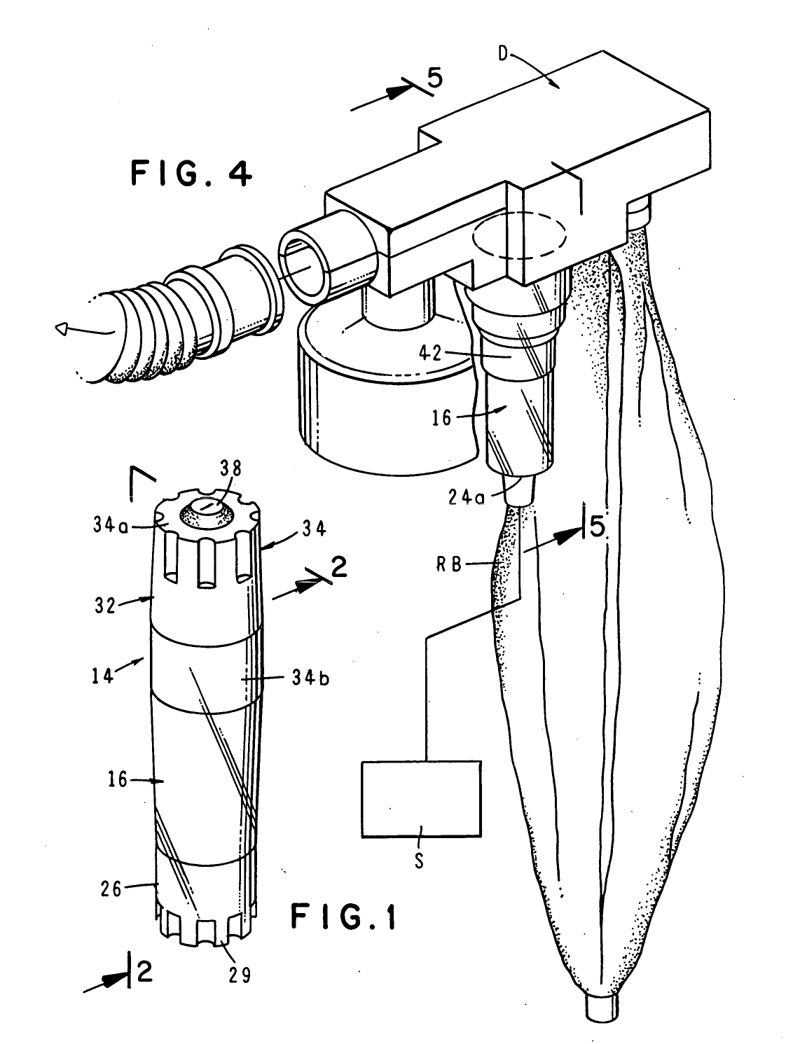

Referring to the drawings and particularly to FIGS. 1 through 3, one form of the miniaturized jet nebulizer unit of the invention for delivering a multiplicity of particles of aerosolized medication of a selected size to a patient is there illustrated and generally designated by the 14. As will be discussed more fully hereinafter, a novel feature of the nebulizer unit of the invention resides in the fact that it can be supplied pre-filled with the required inhalable liquid medication, used for a single treatment, and then discarded.

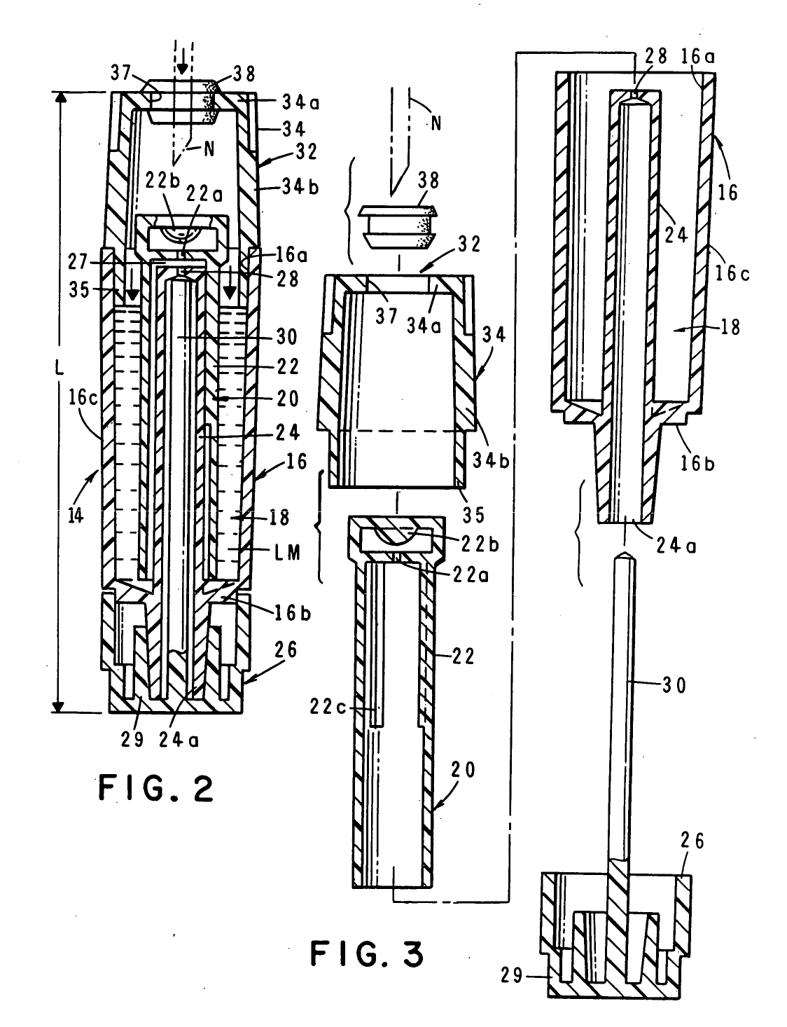

As previously mentioned, nebulizer 14 is quite small and preferably, but not limitedly, has an overall length “L” of between about 2.0 and about 3.0 inches (FIG. 2). Uniquely, nebulizer 14 serves as both the single dose package for the medication to be delivered to the patient and in a manner presently to be described, as a means for converting the liquid medication to a respirable aerosol. In practice, the nebulizer unit 14 can be produced from a commercial polymer in very high quantities by multi-cavity thermoplastic injection molding techniques of a character well understood by those skilled in the art. The number of components that make up the nebulizer unit is intentionally minimized to facilitate molding and to enable automatic robotic assembly and testing. Inasmuch as the nebulizer unit is discarded after each treatment, its use negates the need for extensive preparation and filling prior to the treatment by healthcare professionals or home care patients. Further, because it is intended to be discarded after use, there is no need for nebulizer cleaning thereby eliminating this time-consuming step and removing any doubt about the quality and effectiveness of the cleaning process. These novel qualities of the nebulizer unit of the invention serve to significantly reduce the immense amount of professional labor time by hospital respiratory care departments and at the same time substantially reduce the possibility of iatrogenic cross-contamination via improperly cleaned nebulizers. For the pharmaceutical company, the unique design of the nebulizer unit of the invention provides a higher margin of medication safety because the need for having either a healthcare practitioner or a home care patient transfer a drug from its packaged container into the nebulizer unit is eliminated. The deliberate integral capacity limitation of the nebulizer unit for any given drug also mitigates against unauthorized admixture of the self-contained drug with other agents.

Referring particularly to FIGS. 1 through 3 of the drawings, the nebulizer unit 14 of the invention here comprises a central body 16 having first open end 16a, a second end 16b and a tapered sidewall 16c. Tapered sidewall 16c defines a fluid reservoir 18 for containing a single dose of between about 2 and about 4 milliliters of aerosolizable liquid medicament “LM”. As indicated in FIG. 11, central body 16 has a diameter “DIA” of between about 0.5 and about 0.8 inches.

Disposed within reservoir 18 for converting the aerosolizable liquid medicament into an aerosolized medication is a nebulizer assembly 20 that includes a moldable plastic nebulizer body 22 having a nebulizer orifice 22a and a deflector element 22b (FIGS. 2 and 3). Mounted within central body 16 is an elongated fluid flow tube 24 that forms a part of the nebulizer assembly of the present invention and includes a gas inlet port 24a and a gas outlet port 28 that is in communication with nebulizer orifice 22a.

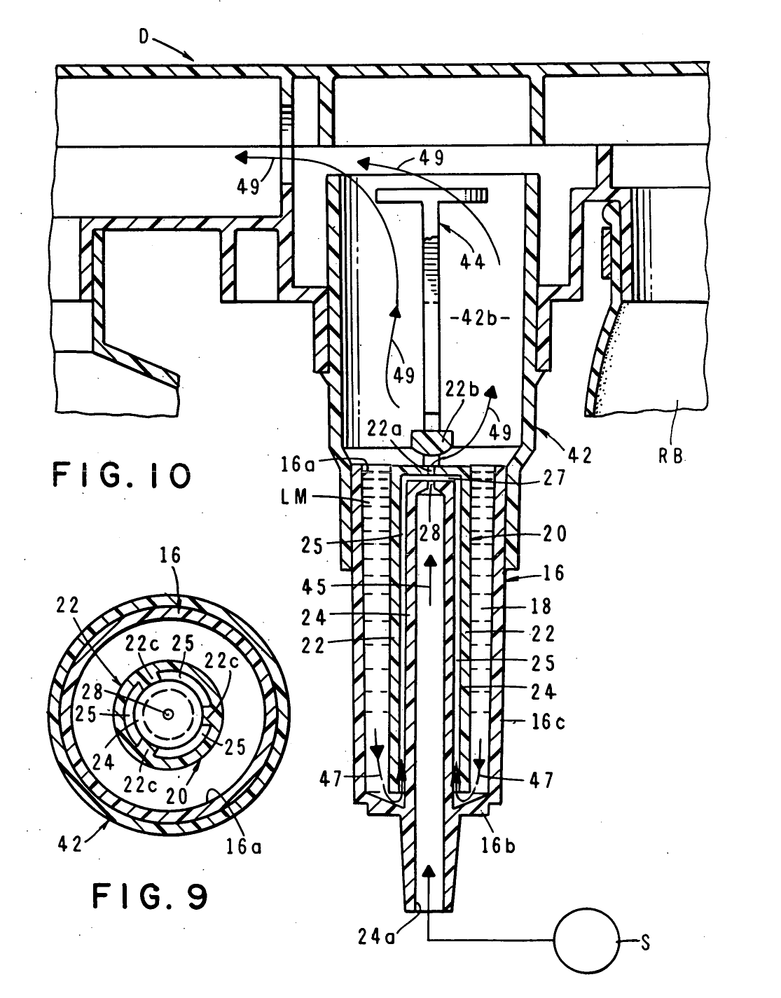

As best seen by referring to FIGS. 2, 3 and 9, nebulizer body 22 is telescopically receivable over flow tube 24 and includes a plurality of circumferentially spaced ribs 22c that cooperate with the outer wall of the flow tube to define a plurality of fluid flow paths 25 (FIG. 9). When the nebulizer body is in position over the flow tube, the components cooperate to define a transverse fluid passageway 27 that is in communication with the plurality of fluid flow passageways 25 and with gas outlet port 28. With this construction, when the reservoir 18 is filled with the aerosolizable liquid medicament “LM” and when the fluid flow tube 24 is interconnected with a source of gas under pressure “S” (FIG. 4), the aerosolizable liquid medicament “LM” will, in a manner presently to be described, be aerosolized to produce a multiplicity of particles of aerosolized medication.

Removably connected to central body 16 is a bottom closure assembly 26 that includes a supporting base 29 and an elongated stem 30 that is connected to supporting base 29 in the manner best seen in FIG. 3 of the drawings. As indicated in the drawings, elongated stem 30 is telescopically, sealably receivable within the fluid flow tube 24 for sealing the gas inlet port 24a thereof. In one form of the present invention, supporting base 29 functions to enable proper positioning of nebulizer 14 for automated robotic filling procedures. In this regard, it should be noted that the overall design of the nebulizer unit of the present invention is such that it is fully compatible with an automated robotic assembly process, with automated robotic post-assembly functional testing and quality assurance inspection, and with automatic robotic packaging processes for packaging and shipping the assembled unit in a fashion that meets the needs of the pharmaceutical companies.

Removably connected to first open end 16a of central body 16 is a top closure assembly 32 that comprises a part of the fill means of the invention for filling reservoir 18 with a suitable liquid medicament. Top closure assembly 32 functions to close the first open end of the central body and also functions to enable the reservoir 18 to be filled with the aerosolizable liquid medicament “LM”. As best seen in FIG. 3 of the drawings, assembly 32 comprises a closure cap 34 that includes a top wall 34a and a tapered skirt portion 34b that is connected to the top wall and depends therefrom. Tapered skirt portion 34b includes a reduced diameter portion 35 that is sealably receivable within open end 16a of central body portion 16 in the manner illustrated in FIG. 2 of the drawings. This is but one form of closure that was designed into the working prototype to demonstrate proof of concept. Many other forms of closure are contemplated and the unit is intentionally made adaptable to different closure methodologies as will be required by different pharmaceutical companies.

Top wall 34a of the closure 34 is provided with an aperture 37 that sealably receives an elastomeric plug 38. As indicated in FIG. 3, aperture 37 can be traversed by the needle “N” of the automated filling apparatus (not shown) that contains the liquid medicament that is to be used to fill the reservoir 18. This is but one form of filling that was designed into the working prototype to demonstrate proof of concept. Many other forms of filling are contemplated based upon the variety of automated filling machinery presently available in the pharmaceutical industry.

After receipt of the requisite number of units by the pharmaceutical company, the units can be filled with a suitable liquid medicament by means of an automated robotic filling process, as previously mentioned, thereby rendering them “pre-filled” in the perspective of the end-user.

Elongated stem 30 effectively seals elongated fluid tube 24 against leakage of liquid medication through 28 after filling and during any subsequent transport of the packaged pre-filled nebulizers before they are used. Immediately prior to use, bottom closure assembly 26 is manually twisted and removed thereby withdrawing the stem 30 from the fluid tube 24 thus readying the device for use. Bottom closure assembly 26 is now discarded.

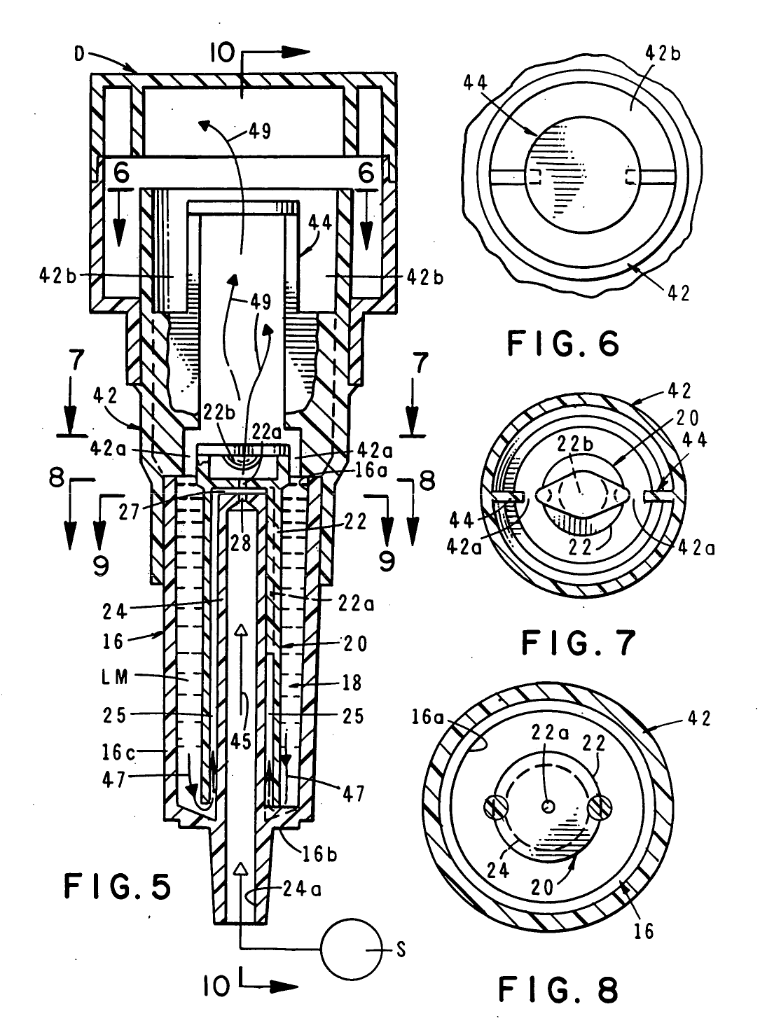

Turning now to FIG. 5 of the drawings, when the nebulizer unit of the present invention is to be used with a dosimetric patient delivery device “D”, such as that described in U.S. Pat. No. 5,727,542 issued to one of the present inventors, the bottom closure assembly 26 is removed and discarded, then the top closure assembly 32 is removed from the central body portion 16 and an injection molded connector adapter 42 is connected to the central body portion in the manner illustrated in FIGS. 5 and 10 of the drawings. Connector adapter 42 includes inlet ports 42a that are in communication with an expansion chamber 42b for expanding the plume of driving gas and decelerating the multiplicity of particles of aerosolized medication emitted from the nebulizer orifice 22a.

As best seen in FIGS. 5, 7 and 10 of the drawings, connector adapter 42 further includes an internal baffle assembly 44 for reducing the size of the multiplicity of particles of aerosolized medication reaching the patient delivery device. In this regard, the volume of expansion chamber 42b must be sufficiently large to enable the aerosol-laden gas plume emitting from the nebulizer orifice 22a to sufficiently re-expand and for the multitude of aerosol particles produced to decelerate in order that the larger particles deliberately encounter the baffling effects of the device and recombine into liquid droplets which are recycled through the nebulizer while the smaller, respirable, particles are effectively emitted from the nebulizer and carried forward to the patient by the gas flow through the nebulizer.

While in the preferred embodiment of the invention, the expansion, or deceleration, chamber 42b is provided as a separate component that can be conveniently attached to the selected patient delivery interface in series with the nebulizer unit, and the deceleration chamber can, for particular end use requirements, be incorporated into the basic nebulizer unit.

The dosimetric patient delivery device “D”, such as that described in U.S. Pat. No. 5,727,542, when coupled with the nebulizer 14 in a manner illustrated in FIGS. 4, 5 and 10 of the drawings, provides the ability to semi-quantitize the patient dose and deliver a drug with such efficiency that often the patient inhalation time to receive a required dose of medication can be reduced to a fraction of that now required using prior art inhalation devices. This novel combination delivers superior patient dose consistency and repeatability even over a wide range of patient breathing parameters. It also provides the ability to accurately predict the actual dose delivered to the patient within ±20% of that predicted when using the dose quantification equation presently to be discussed. Because of its pertinence, U.S. Pat. No. 5,727,542 is hereby incorporated by reference as though fully set forth herein.

Operationally, the device described and incorporated by reference Patent '542 includes a novel, almost resistance-free flapper valve mechanism which directs the output of nebulizer 14 to the patient upon inhalation, and into a reservoir bag “RB” during the period of patient exhalation (FIG. 4). That aerosol which is temporarily retained in the bag becomes additional medication for the patient upon the next inhalation and supplements the delivery of medication provided by real-time operation of the nebulizer rather than being shunted out through the expiratory pathway. Typically in conventional, prior art nebulizer devices, the medication aerosolized during the patient's expiratory phase is lost to the atmosphere and essentially wasted. The immediate obvious benefits of the system of the present invention as illustrated in FIGS. 4, 5 and 10 of the drawings are: (1) drug delivery efficiency is increased dramatically (on average by a factor of 2.4 times); (2) far less medication is wasted by release to the atmosphere; (3) if oxygen is used as the driving gas to power the nebulizer, the fraction of inspired oxygen (FIO₂) provided to the patient will be maintained at a high level.

A less-obvious secondary benefit of the invention resides in the more consistent and reproducible dosing quantities to the patient. If the actuating flow of oxygen or air to the nebulizer unit is in the region of 6 or 7 liters per minute (L/min), and if the patient's minute ventilation (tidal volume multiplied by respiratory rate) is essentially the same as the actuating flow, use of the system of the present invention minimizes very greatly, changes in drug delivery due to differing breathing patterns. Inasmuch as these operating parameters closely match typical human breathing patterns, this system will accommodate a range of patients from pediatrics through adults. In this regard, experience has shown that the system, when used with adult or semi-adult patients, will maintain dose repeatability to within ±20%.

Further, from the Dose Quantification equation presently to be discussed, it is obvious that the patient delivered dose of medication is directly proportional to the drug concentration (mg/mL) being aerosolized and the treatment time, all other factors being constant. Therefore, by proper selection of the drug concentration in the pre-filled nebulizer unit, and regulation of the treatment time, the desired doses can be delivered to the patient in as little as one-minute of treatment time.

In using the apparatus of the invention in connection with a dosimetric patient delivery device “D”, the top closure assembly 32 is disconnected from the body portion 16 and the connector adapter 42 is interconnected with the inlet port of the dosimetric patient delivery device “D” in the manner illustrated in FIGS. 5 and 10 of the drawings. This done, the bottom closure assembly 26 is removed from the elongated fluid flow tube 24 thereby exposing the gas inlet port 24a. Next, the fluid flow tube 24 is interconnected with the source of gas under pressure “S” (FIG. 4). The gas is preferably supplied to the nebulizer from the source “S” at a flow rate of about 6 to 7 liters per minute. As illustrated in FIG. 5, the gas flowing through the gas inlet port 24a in the direction of the arrow 45 passes through the very small nebulizer orifice 28 provided in the nebulizer body 24. As the gas courses upwardly through the fluid flow tube 24, it creates a partial vacuum in the circumferentially spaced fluid passageways 25. This vacuum causes the level of the liquid medicaments in the reservoir 18 to flow into passageways 25 in the direction of the arrow 47 and then to flow over the top of the fluid passageways 25. Due to the basic design of the nebulizer and in accordance with the Bernoulli effect, when the stream of gas flowing through the fluid flow tube strikes the liquid drawn from the reservoir it will be predictably converted into a fine mist containing a mixture of particles of aerosolized medication of varying sizes that will be carried upwardly through spray orifice 22a formed in plastic nebulizer body 22. In the present form of the invention, the nebulizer orifice produces a multiplicity of particles comprising larger particles of a size exceeding 5 microns and smaller particles of a size between 0.2 to 5 microns.

After flowing through orifice 22a, the fine particulate-laden mist following impact with the selector element 22b will flow into expansion chamber 42b of the connector adapter 42 and around and about baffle 44 in the direction of the arrows 49 in a manner to decelerate the multiplicity of particles of aerosolized medication emitted from the nebulizer orifice 22a. This deceleration of the particles reduces the size of the particles reaching the outlet port of the device and limits the size of the particles that ultimately reach the patient.

Through use of the combination nebulizer and dosimetric patient delivery device “D” as described in the preceding paragraphs, the following Dose Quantification equation can be effectively used in predicting the delivered patient dose [Inhaled Mass, predicted (IMp)]:

IMp=C×AGR×K×SE×T

where:

IMp: Inhaled Mass, predicted (mg)

C: Drug Concentration (mg/mL)

AGR: Aerosol Generation Rate, i.e., the rate of conversion of liquid to aerosol, (mL/min)

K: AGR Constant; fractional multiplier representing the typical drug content as fraction of AGR

SE: System Efficiency; fractional multiplier representing the System Efficiency (i.e., percentage of output drug/nebulizer output)

T: Time of aerosolization, i.e., Treatment Time, (minutes)

### Example

Calculate predicted dose to patient when aerosolizing a medication, such as albuterol, with a concentration [C] of 5 mg/mL for one minute with an AGR of 0.25 and a constant [K] of 0.5 and SE of 0.65.

IMp=5×0.25×0.5×0.65×1=0.32 mg

An Inhaled Mass (delivery) of 0.32 mg of albuterol is typical of the nominal dose of albuterol delivered by most conventional prior art small volume nebulizers.

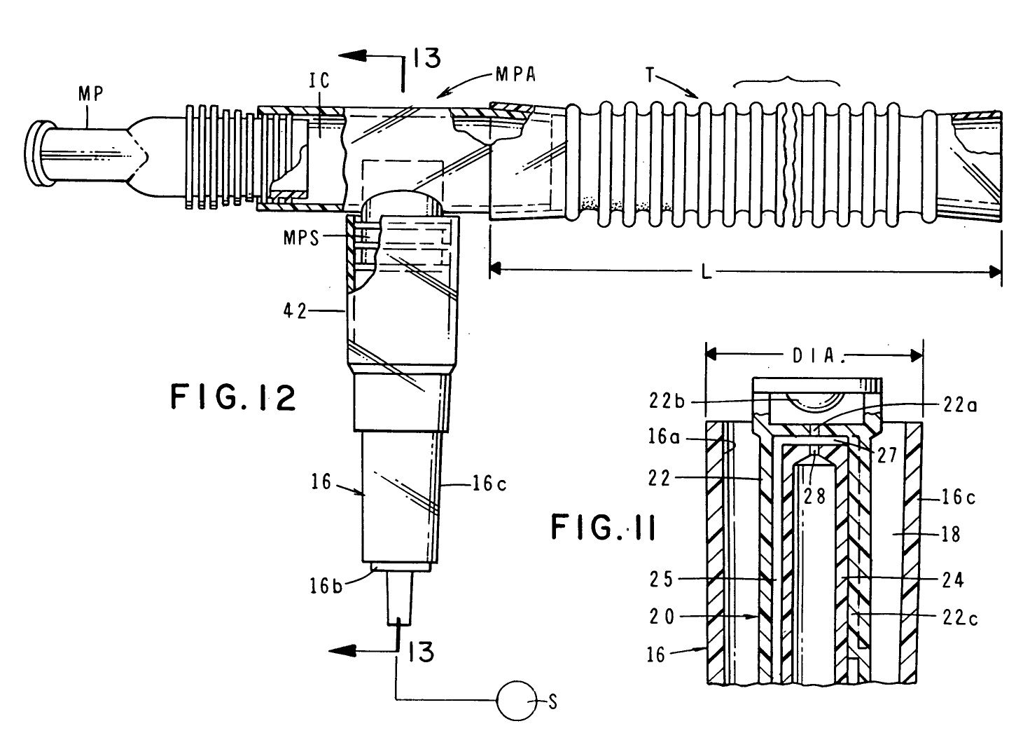

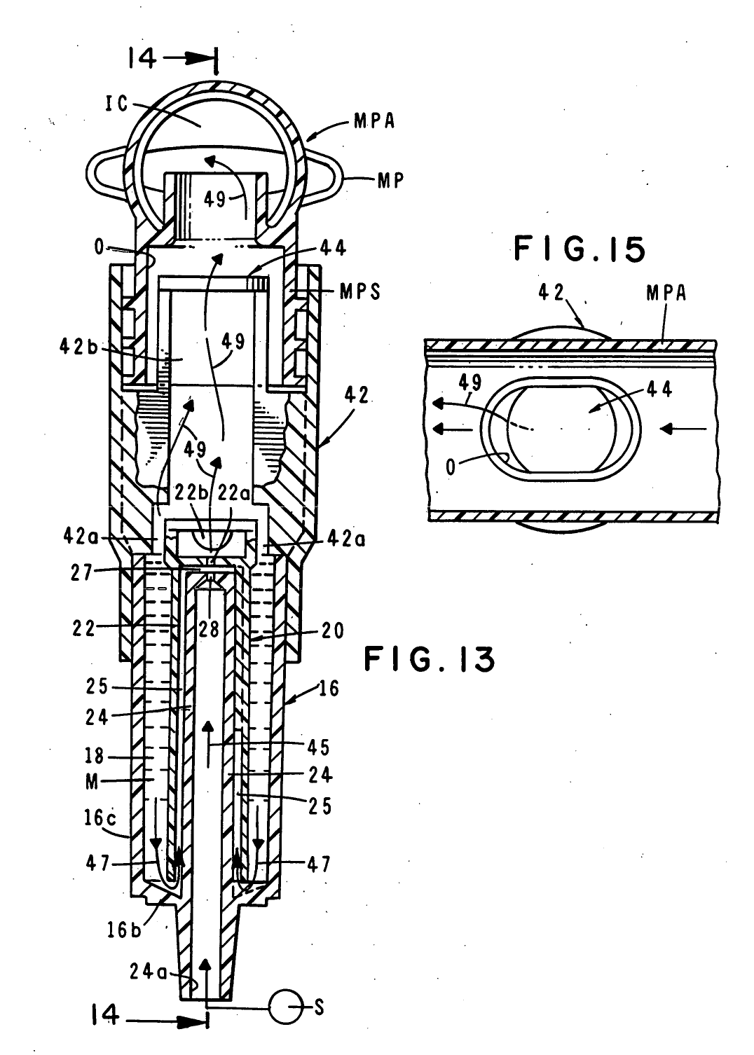

Turning now to FIGS. 12, 13 and 14, the nebulizer unit of the invention is there shown interconnected with a different form of patient delivery device, here shown as a conventional mouthpiece and tee connector assembly “MPA” that comprises a corrugated aerosol reservoir flex tubing “T” having a length “L” and a conventional mouthpiece “MP”.

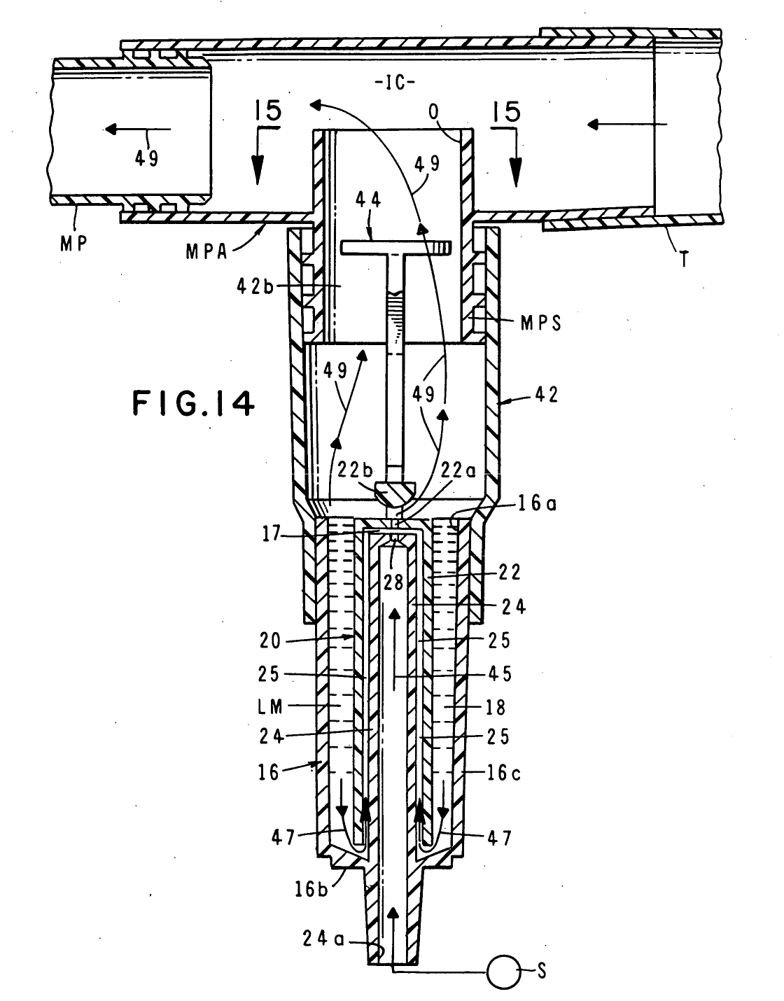

As best seen in FIG. 14, assembly “MPA” is provided with a skirt “MPS” having inlet opening “O”. To interconnect the nebulizer unit of the invention with the mouthpiece assembly, the connector adapter 42 is telescopically received over the skirt “MPS” in the manner depicted in FIG. 14.

Following removal of the lower closure assembly 26, the fluid flow tube 24 is interconnected with the source of gas under pressure “S” (FIGS. 12, 13 and 14). As illustrated in FIG. 14, the gas flowing through the gas inlet port 24a in the direction of the arrow 45 passes through the very small nebulizer orifice 28 provided in the nebulizer body 24. As before, as the gas courses upwardly through the fluid flow tube 24 it creates a partial vacuum in the circumferentially spaced fluid passageways 25. This vacuum causes the level of the liquid medicaments in the reservoir 18 to flow into passageways 25 and then to flow over the top of the fluid passageways 25. Due to the basic design of the nebulizer, when the stream of gas flowing through the fluid flow tube strikes the liquid drawn from the reservoir, it will be predictably converted into a fine mist containing a mixture of particles of aerosolized medication of varying sizes that will be carried upwardly through spray orifice 22a formed in plastic nebulizer body 22.

After flowing through orifice 22a, the fine particulate-laden mist following impact with the selector element 22b will flow into expansion chamber 42b of the connector adapter 42 and around and about baffle 44 in the direction of the arrows 49 in a manner to decelerate the multiplicity of particles of aerosolized medication emitted from the nebulizer orifice 22a. The particles of aerosolized medication will then flow to the internal chamber “IC” of the mouthpiece, along the length of the mouthpiece, outwardly of the mouthpiece outlet and into the mouth of the patient.

By way of summary, the several advantages of the apparatus of the present invention for hospitals and home care agencies include the following:

1. Patient delivery time using the combination nebulizer 14 and dosimetric patient delivery device “D” for the delivery of albuterol and similar inhalable drugs can be reduced to about a one-minute treatment time.
2. Influence of patient breathing pattern on drug delivery can be substantially minimized or negated.
3. Single-use “throw-away” technology embodied in the apparatus of the invention completely eliminates the need for post-treatment nebulizer cleaning and removes all doubt about the effectiveness of this procedure.
4. Within limits, through use of the apparatus of the invention, patient dose is reasonably quantifiable and predictable.
5. Total time for hospital patient treatments can be greatly reduced, through reduction of both pre-treatment set-up time and post-treatment clean-up time, thereby resulting in both labor-savings and cost-savings for the facility.
6. The use of the apparatus of the invention, in combination with the dosimetric patient delivery device “D,” substantially reduces atmospheric contamination to less than approximately 15% of nebulizer loading dose.
7. The very short treatment time of approximately one minute contributes to improved patient compliance, especially with patients receiving multiple inhalation drugs.
8. Particle size control can be readily built into the nebulizer design; that is, different particle baffling designs can be made available for different desired particle sizes as characterized by measurements of mass median aerodynamic diameter (MMAD).
9. Device acquisition cost to the healthcare facility or home care agency is substantially or completely offset by labor savings in the hospital environment and probable reduction in service or maintenance calls for home care patients undergoing self-treatment in the home environment.

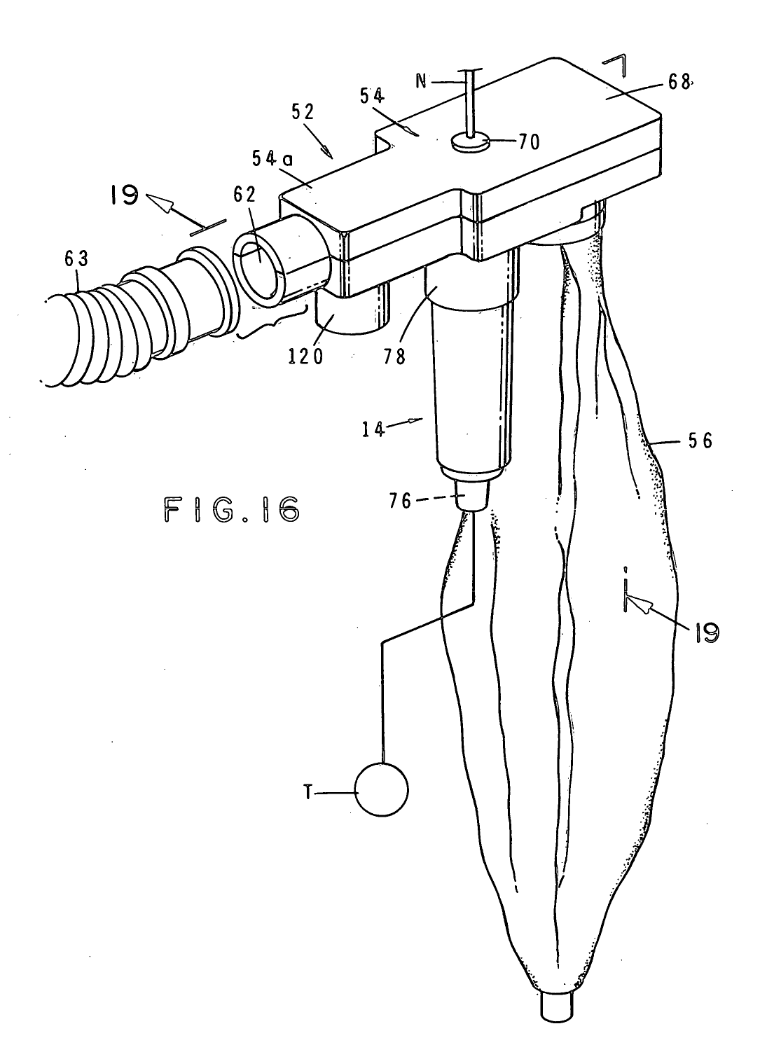

Referring to the drawings and particularly to FIGS. 16, 17 and 19, one alternate form of aerosol inhalation apparatus is there shown and generally designated by the numeral 52. This form of the invention is similar in some respects to the apparatus illustrated and described in FIGS. 1 through 11 and like numerals are used in FIGS. 16 through 22 to identify like components. Apparatus 52 here comprises a sectionalized main housing 54 to which is attached an inflatable bag 56 and nebulizer means, shown here as a nebulizer unit 14 which is substantially identical in construction and operation to that previously described in connection with the embodiment of FIGS. 1 through 11. A first end 54a of the main housing is provided with a standard size breathing port 62 for ready patient interfacing with the aerosol system via a conduit, or breathing tube 63 (FIG. 16). In a manner presently to be described, the various sections of chambers of the main housing are interconnected by appropriately baffled fluid flow passageways.

Turning particularly to FIG. 19, the nebulizing means, or nebulizer unit 14, of this latest embodiment comprises a central body 16 having first open end 16a, a second end 16b and a tapered sidewall 16c. Tapered sidewall 16c defines a fluid reservoir 18 for containing a single dose of between about 2 and about 4 milliliters of aerosolizable liquid medicament “LM”. As before, central body 16 has a diameter of between about 0.5 and about 0.8 inches.

Disposed within reservoir 18 for converting the aerosolizable liquid medicament into an aerosolized medication is a nebulizer assembly 20 that includes a moldable plastic nebulizer body 22 having a nebulizer orifice 22a and a deflector element 22b (FIGS. 2 and 3). Mounted within central body 16 is an elongated fluid flow tube 24 that forms a part of the nebulizer assembly of the present invention and includes a gas inlet port 24a and a gas outlet port 28 that is in communication with nebulizer orifice 22a. As in the earlier described embodiment of the invention, the nebulizer unit can be supplied pre-filled with the required inhalable liquid medication, used for a single treatment, and then discarded.

Carried by a wall 68 of housing 54 which is disposed directly above nebulizer 14, is access means for accessing nebulizer 14 to supply medication fluid to reservoir portion 18 thereof. In the embodiment of the invention shown in FIG. 19 of the drawings, the accessing means comprises a self-sealing, penetrable septum 70 which, as shown in FIGS. 17 and 19, is sealably mounted within an aperture 72 provided in top wall 54 of housing 54. For certain applications, septum 70 can also comprise a split septum adapted to receive a blunt cannula. Septum 70 is preferably constructed of soft rubber or other suitable elastomer material which is readily penetrable by a blunt cannula or by the needle “N” of a hypodermic syringe (FIG. 16).

Nebulizer unit 14 also includes a gas inlet means here comprising a gas inlet port 76, which is interconnected with a source of gas under pressure, such as a tank “T” (FIG. 17). The nebulizer gas inlet means functions to permit the controlled introduction into the nebulizer of a selected gas under pressure in the manner previously described herein, to cause nebulizing of the fluid disposed within reservoir 18 of the nebulizer. The fluid disposed within reservoir 18 can comprise any of a large number of medications, or mixtures thereof, depending upon the end use to be made of the apparatus.

As best seen in FIG. 19 of the drawings, housing 54 includes a downwardly extending connector segment 78 which is adapted to receive the upper portion of tapered sidewall 16c.

Disposed within a first or intermediate chamber 77 of housing 54, is the important airflow baffle 80 of this latest form of the invention. Chamber 77 is located immediately above connector segment 78 and intermediate the second, or rearward housing chamber 82 and the third, or forward housing chamber 84. As will be discussed in greater detail hereinafter, the addition baffle 80 uniquely causes aerosol being drawn from the reservoir bag 56 upon patient inhalation to be accelerated across and around the point of Venturi action in the nebulizer component. This change in air flow creates a reduction in pressure, thereby substantially increasing the rate of fluid aerosolization. By way of example, the addition of this novel airflow baffle incorporates the benefits from both dosimetric/reservoir and breath-enhancement type devices and uniquely decreases patient dosing time to on the order of one to two minutes, when aerosolizing a standard unit dose of Albuterol (2.5 mg/3 m).

By way of brief background, Dr. John Dennis in “Practical Handbook of Nebulizer Therapy”, Martin-Derity, 2004, indicates (Page 15) that there are three jet nebulizer designs: Constant output, Breath-enhanced, and dosimetric. As discussed in the incorporated by reference U.S. Pat. No. 5,727,542, the apparatus described therein incorporates a reservoir bag which retains aerosol medication generated during time of patient exhalation for subsequent use during the next inhalation. As such, since essentially all of the nebulizer output is received by the patient, this device is considered “dosimetric” by Dr. Dennis' definition. Further, quoting Dr. Dennis, “breath-enhanced designs operate by allowing air inhaled by the patient to be drawn through the nebulizer, thus “enhancing” the rate of air and aerosol output from the nebulizer during inhalation. On expiration, the output from the nebulizer falls back to a lower rate”.

As previously mentioned, through the addition baffle 80 to intermediate chamber 77, the apparatus of this latest form of the invention uniquely combines the features of breath-enhancement devices with the dosimetric/reservoir type devices. The present inventor is unaware of any prior art device that incorporates into a single device the desirable features of dosimetric/reservoir and breath-enhancement. Such a clinical device should prove to be extremely beneficial by shortening patient treatment times, thereby overcoming patient non-compliance issues.

In using the apparatus of the invention, the nebulizer unit is interconnected with the mouthpiece assembly by telescopically inserting the upper portion of the tapered sidewall 16c of the nebulizer unit into connecter 76 of the mouthpiece assembly in the manner shown in FIG. 19 of the drawings. Following removal of the lower closure assembly 26 of the nebulizer unit, the fluid flow tube 24 is interconnected with the source of gas under pressure, namely tank T (FIGS. 17 and 19). As illustrated in FIG. 19, the gas flowing through the gas inlet port 76 in the direction of the arrow 83 passes through the very small nebulizer orifice 28 provided in the nebulizer body 24. As before, as the gas courses upwardly through the fluid flow tube 24 it creates a partial vacuum in the circumferentially spaced fluid passageways 25. This vacuum causes the level of the liquid medicaments in the reservoir 18 to flow into passageways 25 and then to flow over the top of the fluid passageways 25. Due to the basic design of the nebulizer, when the stream of gas flowing through the fluid flow tube strikes the liquid drawn from the reservoir it will be predictably converted into a fine mist containing a mixture of particles of aerosolized medication of varying sizes that will be carried upwardly through spray orifice 22a formed in plastic nebulizer body 22.

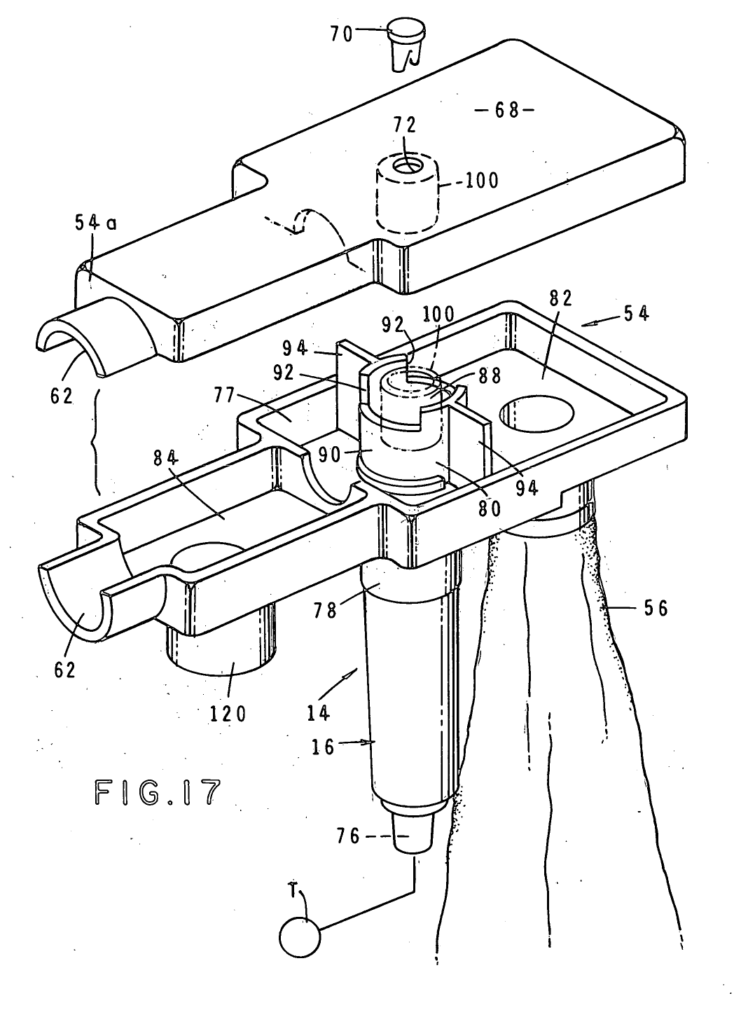

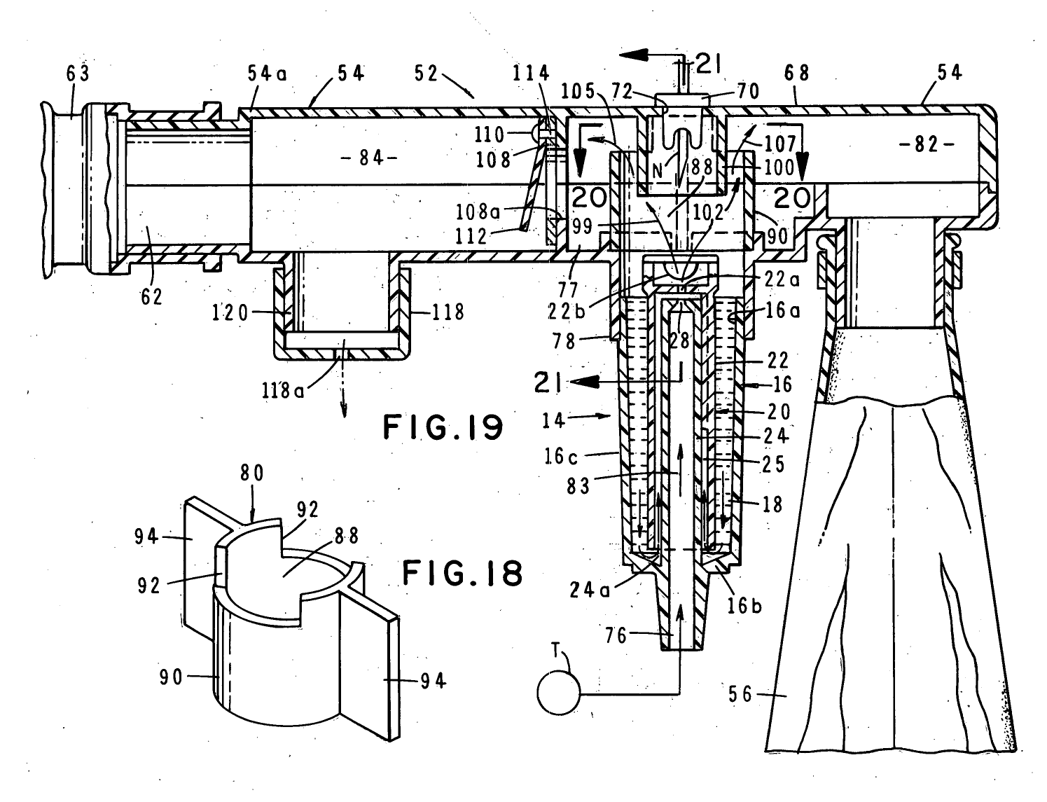

After flowing through orifice 22a, the fine particulate-laden mist following impact with the selector element 22b will flow into expansion chamber 88 of the airflow baffle 80. As best seen by referring to FIG. 18 of the drawings, expansion chamber 88 is defined by a generally cylindrically shaped housing 90 having diametrically opposed flow openings 92 provided in the upper portion thereof. Affixed to opposing sides of housing 90 at about 90 degrees from flow openings 92, are transversely extending baffle walls 94. As illustrated in FIGS. 17 and 20, baffle walls 94 extend across intermediate chamber 77 and one of the diametrically opposed openings 92 communicates with chamber 82 while the other of the diametrically opposed openings 92 communicates with intermediate chamber 77. As best seen in FIG. 19, expansion chamber 88 of the airflow baffle 80 is located immediately above housing connector segment 78 and receives the aerosol spray emitting from the nebulizer in the manner indicated by the arrows 99 of FIG. 21.

Extending downwardly from the top wall of the housing and into chamber 88 of the airflow baffle is a generally cylindrically shaped wall 100. Wall 100 cooperates with wall 90 of the airflow baffle to define a fluid flow path 102 that receives the aerosol spray flowing into expansion chamber 88 in the direction of the arrows 99. As indicated by the arrows 105 and 107 of FIG. 19, the aerosol flows from flow passageways 102 into chambers 77 and 82 via the opposed openings 92 formed in housing 90 of the airflow baffle (see also FIG. 20). It is this novel configuration of the airflow baffle 80 and its strategic positioning within housing chamber 77 that uniquely causes aerosol being drawn from the reservoir bag 56 upon patient inhalation to be accelerated across and around the point of Venturi action in the nebulizer component. This change in air flow creates a reduction in pressure, thereby substantially increasing the rate of fluid aerosolization. As previously discussed herein, the addition of this novel airflow baffle incorporates the benefits from both dosimetric/reservoir and breath-enhancement type devices and uniquely decreases patient dosing time to on the order of one to two minutes when aerosolizing a standard unit dose of Albuterol (2.5 mg/3 m).

As the patient inhales, the aerosol flowing from one of the passageways 102 into chamber 77 in the direction of the arrow 105 (FIG. 19) flows through a passageway 108a formed in a partition wall 108, which separates chambers 77 and 84, through chamber 84 and through breathing tube 63 into the patient's lungs. As best seen in FIG. 19, affixed to wall 108 at a single pivot point 110 is a generally circular substantially flexible flapper valve member 112. Valve member 112 can be constructed of a number of materials including plastic and various yieldably deformable elastomeric materials. Pivot point 110 is defined by a fastener such as rivet 114 which passes through member 112 at a location proximate its outer periphery and then through partition wall 108. With this unique construction, fluid passing through passageway 108a will cause valve member 112 to open in a novel pivoting motion about pivot point 110. Conversely, fluid flowing in an opposite direction will cause valve member 112 to securely close and move into sealing engagement with partition wall 108, thereby blocking fluid flow through passageway 108a.

When the apparatus is in use, patient inhalation will cause valve member 112 to open to permit the aerosolized medication to flow from chamber 77 into chamber 84 and then into the patient's lungs. During the time of patient breath hold and exhalation, the one-way valve will close, causing mist produced during this period of time to be retained within chambers 77 and 82 and within the inflatable bag 56. This accumulation will fully satisfy the patient's next breath tidal volume requirements. Once again, this novel construction permits maximum effective use to be made of the particulate-laden mist being generated by the nebulizer.

In those situations where no harm results from exhalation of the particulate laden mist directly into the atmosphere, a cap 118 which fits securely over a downwardly extending cylindrical portion 120 formed on housing 54 (FIG. 19) is provided, with a small centrally disposed aperture 118a which permits flow to atmosphere and at the same time provides sufficient impedance to such flow as to insure proper closing of valve member 112 upon patient exhalation. When member 112 is closed, aerosol emitting from the nebulizer flows through one of the flow passageways 92 in the direction of the arrow 107 and is deposited in reservoir bag 56.

The next patient inhalation will not only accept newly produced aerosol from the nebulizer, but now pulls aerosol from the reservoir bag 56. Knowing that the rate of patient inhalation greatly exceeds the rate of oxygen/air flow to the nebulizer, orifice opening sizes and physical positioning in relation to the point of Venturi action can be tailored to momentarily change internal baffle air pressure to an extent that aerosol generation rate from the nebulizer will more than double.

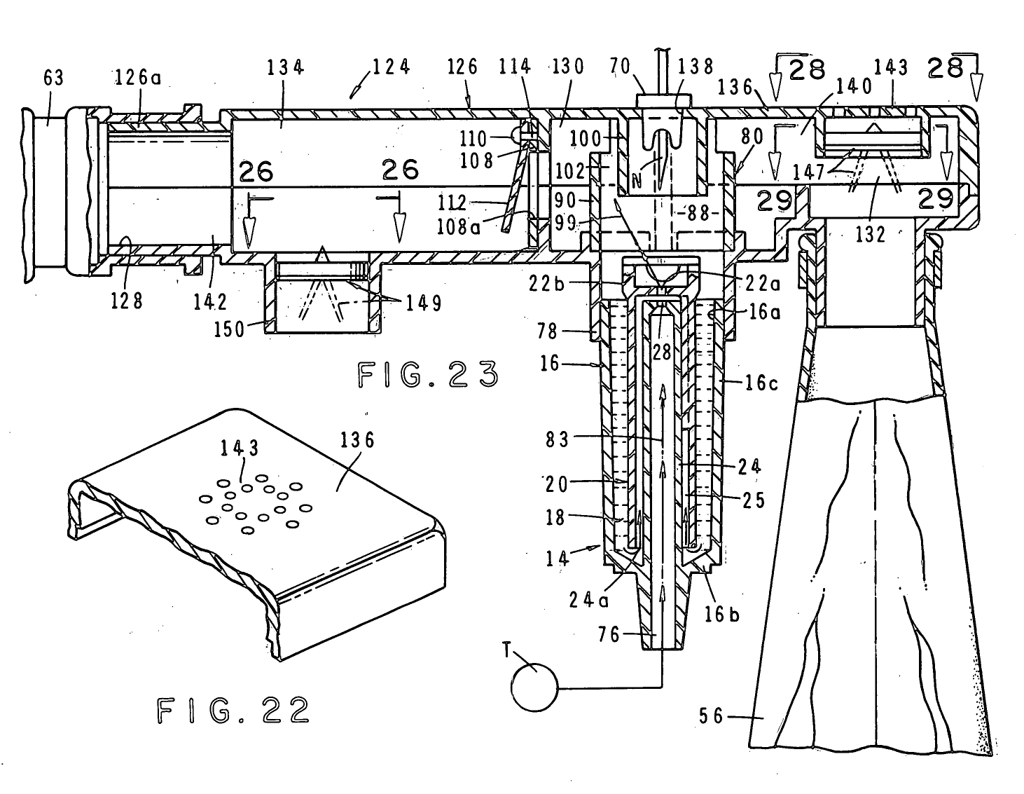

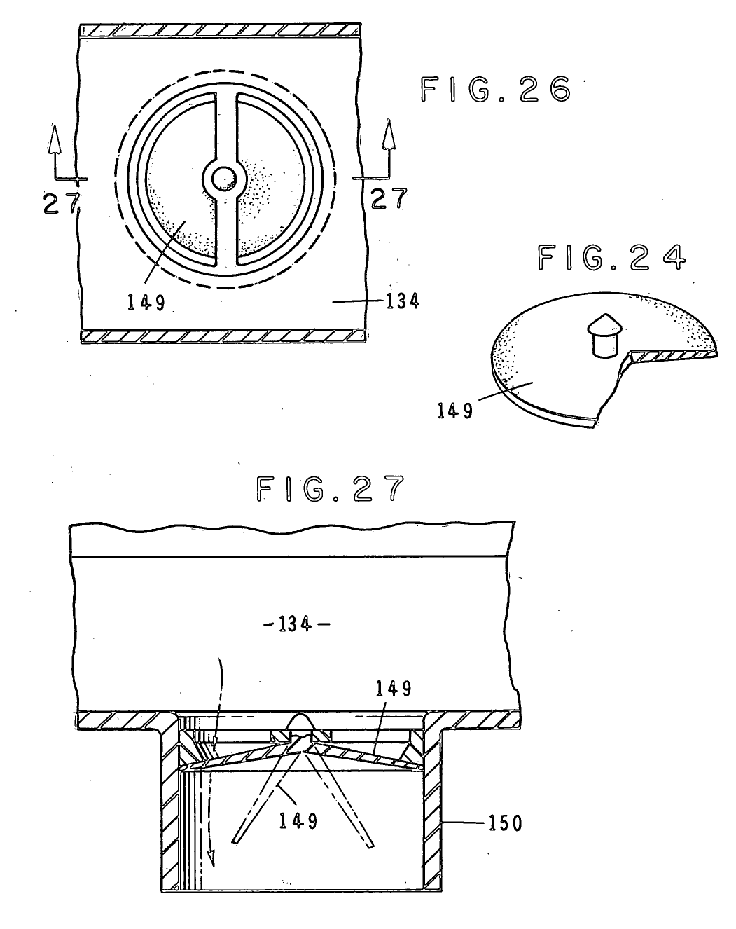

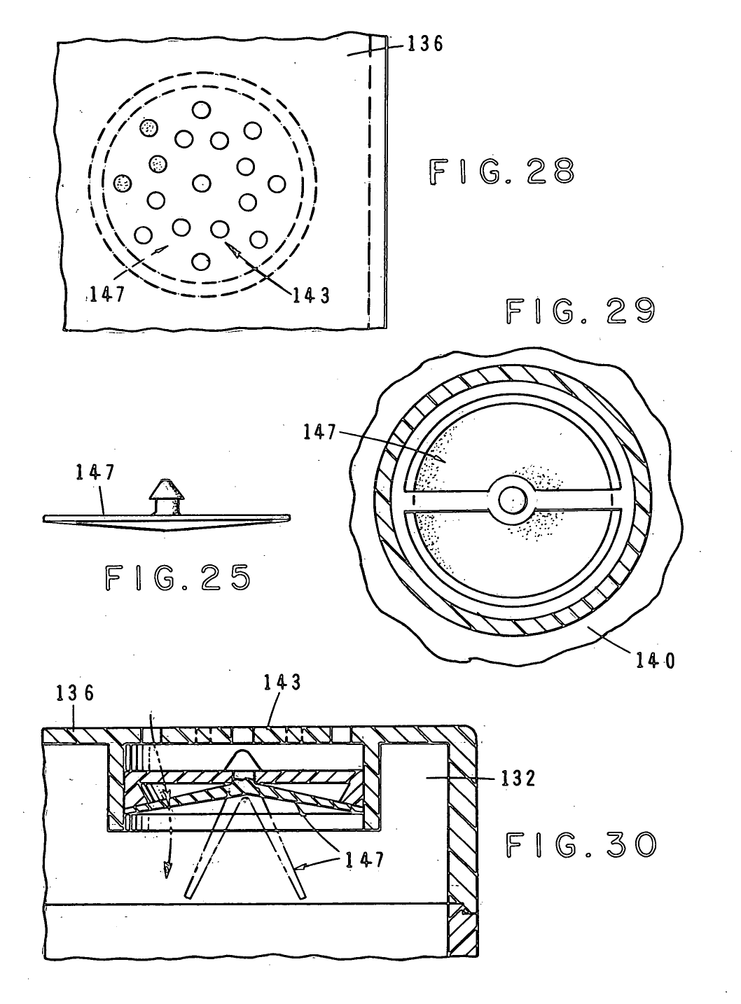

Referring next to FIGS. 23 through 30, still another form of aerosol inhalation apparatus of the invention is there shown and generally designated by the numeral 124. This form of the invention is similar in some respects to the apparatus illustrated in FIGS. 16 through 22 and like numerals are used in FIGS. 23 through 30 to identify like components. The main difference between this latest form of the invention and the earlier described embodiments comprises the addition of two one-way, umbrella type check valves to the sectionalized main housing 126. The purpose of these check valves will presently be described.

Attached to sectionalized main housing 126 is an inflatable bag 56 which is substantially identical in construction and operation to that previously described. Also attached to sectionalized main housing 126 is a nebulizer means, shown here as a nebulizer unit 14 which is also substantially identical in construction and operation to that previously described.

In this latest embodiment of the invention, a first end 126a of the main housing is provided with a standard size breathing port 128 for ready patient interfacing with the aerosol system via a conduit, or breathing tube 63 (see also FIG. 16). As before, the various chambers 130, 132 and 134 of the main housing are interconnected by appropriately baffled fluid flow passageways.

As in the earlier described embodiments of the invention, the nebulizer unit 14 can be pre-filled with the required inhalable liquid medication, used for a single treatment, and then discarded. Carried by a wall 136 of main housing 126, which is disposed directly above nebulizer 14, is access means for accessing nebulizer 14 to supply medication fluid to reservoir portion 18 thereof. As before, the accessing means comprises a self-sealing, penetrable septum 70 which, as shown in FIG. 23, is sealably mounted within an aperture 138 provided in top wall 136 of housing 126.

Disposed within a first, or intermediate chamber 130 of housing 126, is the important airflow baffle 80 of this latest form of the invention. Airflow baffle 80 is also substantially identical in construction and operation to that previously described and uniquely causes aerosol being drawn from the reservoir bag 56 upon patient inhalation to be accelerated across and around the point of Venturi action in the nebulizer component. Chamber 130 is located immediately above the nebulizer connector segment 78 and intermediate the second, or rearward housing chamber 140 and the third, or forward housing chamber 142 to which the breathing tube 63 is connected.

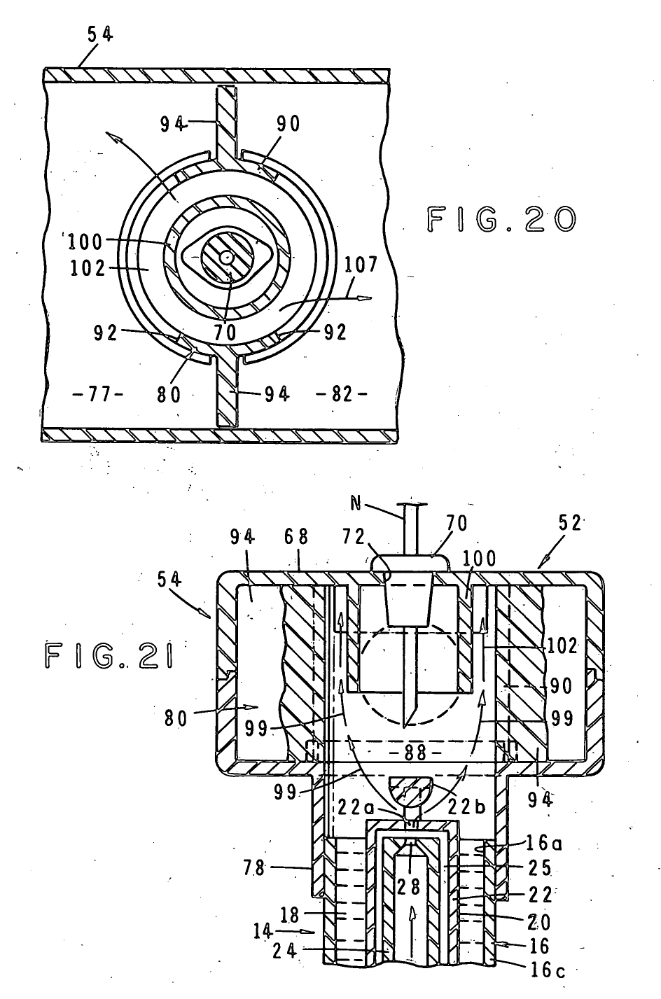

Referring particularly to FIG. 23, it is to be observed that there are three possible sources of air/oxygen for fulfilling the lung capacity requirement of the patient. These include (1) the gas driving the nebulizer 14, (2) medicated air/oxygen from the reservoir bag 56, and (3) possible room air, drawn inwardly through exit port 143 provided in top wall 136 of housing 126. (FIGS. 22 and 23) Knowing that momentary breath inhale requirements can reach a rate of perhaps 20 liters per minute (LPM) or greater, it is obvious that upon occasion there can be air-entrainment through the exit port 143. Any such air going to the patient reduces the velocity of air/oxygen drawn from the reservoir bag, thereby decreasing the desirable breath enhancement features. As illustrated in FIG. 23, a one-way valve umbrella type check valve 147 placed proximate the exit port 143 eliminates this possibility of air entrainment. Placement of a second one way umbrella type check valve 149 in the outlet port 150 provided in forward chamber 134 is such that if needed to fulfill patient momentary requirements, room air can be entrained. This air, however, will be added to that drawn from reservoir bag 56, further increasing the desirable breath enhancement features.

Having now described the invention in detail in accordance with the requirements of the patent statues, those skilled in this art will have no difficulty in making changes and modifications in the individual parts or their relative assembly in order to meet specific requirements or conditions. Such changes and modifications may be made without departing from the scope and spirit of the invention, as set forth in the following claims:

1. A single dose nebulizer unit for delivering a multiplicity of particles of aerosolized medication of a selected size to a patient comprising:
(a) a nebulizer housing having a top wall, spaced apart side walls and first, second and third chambers, said first chamber being in communication with said second and third chambers;
(b) a nebulizer assembly connected to said nebulizer housing and in communication with said first chamber for converting said aerosolizable liquid medicament into an aerosolized medication and for introducing said aerosolized medication into said first chamber, said nebulizer assembly comprising:
(i) a nebulizer body having a first open end, a second end and a nebulizer orifice; and
(ii) a fluid flow tube connected to said second end of said central body, said fluid flow tube having a gas inlet port and a gas outlet port in communication with said nebulizer orifice for aerosolizing said aerosolizable liquid medicament to produce a multiplicity of particles of aerosolized medication; and
(c) an airflow baffle disposed within said first chamber, said airflow baffle comprising:
(i) a baffle housing having flow openings provided therein and including an expansion chamber in communication with said nebulizer; and
(ii) a pair of transversely extending baffle walls connected to said generally cylindrically shaped housing and to said side walls of said nebulizer housing.

2. The nebulizer unit as defined in claim 1 further including an inflatable bag connected to said nebulizer housing and in communication with said second chamber.

3. The nebulizer unit as defined in claim 1 in which said top wall of said nebulizer housing is provided with access means for accessing said nebulizer assembly to supply medication thereto.

4. The nebulizer unit as defined in claim 1 in which said nebulizer housing includes a breathing port in communication with said third chamber.

5. The nebulizer unit as defined in claim 1 in which said nebulizer housing further includes a partition wall separating said first and third chambers, said partition wall having an opening and carrying a flexible flapper valve member for controlling fluid flow through said opening.

6. The nebulizer unit as defined in claim 1 in which said nebulizer assembly produces a multiplicity of particles comprising larger particles of a size exceeding 5 microns and smaller particles of a size between 0.2 to 5 microns.

7. The nebulizer unit as defined in claim 1 in which the overall length of said nebulizer assembly is between about 2.0 and about 3.0 inches.

8. The nebulizer unit as defined in claim 1 in which said nebulizer housing further includes an outlet port in communication with said third chamber.

9. The nebulizer unit as defined in claim 8 further including an umbrella check valve connected to said nebulizer housing for controlling fluid flow through said outlet port in communication with said third chamber.

10. The nebulizer unit as defined in claim 9 in which said nebulizer housing further includes an inlet port in communication with said second chamber.

11. The nebulizer unit as defined in claim 9 further including an umbrella check valve connected to said nebulizer housing for controlling fluid flow through said inlet port in communication with said second chamber.

12. A single dose nebulizer unit for delivering a multiplicity of particles of aerosolized medication of a selected size to a patient comprising:
(a) a nebulizer housing having a top wall, spaced apart side walls and first, second and third chambers, said first chamber being in communication with said second and third chambers;
(b) a nebulizer assembly connected to said nebulizer housing and in communication with said first chamber for converting said aerosolizable liquid medicament into an aerosolized medication and for introducing said aerosolized medication into said first chamber, said nebulizer assembly comprising:
(i) a nebulizer body having a first open end, a second end and a nebulizer orifice; and
(ii) a fluid flow tube connected to said second end of said central body, said fluid flow tube having a gas inlet port and a gas outlet port in communication with said nebulizer orifice for aerosolizing said aerosolizable liquid medicament to produce a multiplicity of particles of aerosolized medication; and
(c) an airflow baffle disposed within said first chamber, said airflow baffle comprising:
(i) a generally cylindrically shaped housing having an expansion chamber in communication with said nebulizer and including an upper portion, said upper portion having diametrically opposed flow openings provided therein; and
(ii) a pair of transversely extending baffle walls connected to said generally cylindrically shaped housing and to said side walls of said nebulizer housing;
(d) an inflatable bag connected to said nebulizer housing and in communication with said second chamber; and
(e) a breathing tube connected to said nebulizer housing and in communication with said third chamber.

13. The nebulizer unit as defined in claim 12 in which said top wall of said nebulizer housing is provided with access means for accessing said nebulizer assembly to supply medication thereto.

14. The nebulizer unit as defined in claim 12 in which said nebulizer housing further includes a partition wall separating said first and third chambers, said partition wall having an opening and carrying a flexible flapper valve member for controlling fluid flow through said opening.

15. The nebulizer unit as defined in claim 12 in which said nebulizer housing further includes an outlet port in communication with said third chamber.

16. The nebulizer unit as defined in claim 15 further including an umbrella check valve connected to said nebulizer housing for controlling fluid flow through said outlet port in communication with said third chamber.

17. The nebulizer unit as defined in claim 16 in which said nebulizer housing further includes an inlet port in communication with said second chamber.

18. The nebulizer unit as defined in claim 17 further including an umbrella check valve connected to said nebulizer housing for controlling fluid flow through said inlet port in communication with said second chamber.

19. A single dose nebulizer unit for delivering a multiplicity of particles of aerosolized medication of a selected size to a patient comprising:
(a) a nebulizer housing having a top wall, spaced apart side walls and first, second and third chambers, said first chamber being in communication with said second and third chambers, said nebulizer housing further including a generally cylindrically shaped wall extending downwardly from said top wall and a partition wall separating said first and third chambers, said partition wall having an opening;
(b) a flexible flapper valve member carried by said partition wall for controlling fluid flow through said opening in said partition wall;
(c) a nebulizer assembly connected to said nebulizer housing and in communication with said first chamber for converting said aerosolizable liquid medicament into an aerosolized medication and for introducing said aerosolized medication into said first chamber, said nebulizer assembly comprising:
(i) a nebulizer body having a first open end, a second end and a nebulizer orifice; and
(ii) a fluid flow tube connected to said second end of said central body, said fluid flow tube having a gas inlet port and a gas outlet port in communication with said nebulizer orifice for aerosolizing said aerosolizable liquid medicament to produce a multiplicity of particles of aerosolized medication; and
(d) an airflow baffle disposed within said first chamber, said airflow baffle comprising:
(i) a generally cylindrically shaped housing having an expansion chamber in communication with said nebulizer and including an upper portion, said upper portion having diametrically opposed flow openings provided therein; and
(ii) a pair of transversely extending baffle walls connected to said generally cylindrically shaped housing and to said side walls of said nebulizer housing;
(e) an inflatable bag connected to said nebulizer housing and in communication with said second chamber;
(f) a breathing tube connected to said nebulizer housing and in communication with said third chamber; and
(g) access means provided in said top wall of said nebulizer housing for accessing said nebulizer assembly to supply medication thereto.

20. The nebulizer unit as defined in claim 19 in which said nebulizer housing further includes:
(a) an outlet port in communication with said third chamber;
(b) an umbrella check valve disposed proximate said outlet port for controlling fluid flow through said outlet port in communication with said third chamber;
(c) an inlet port in communication with said second chamber; and
(d) an umbrella check valve disposed proximate said inlet port for controlling fluid flow through said inlet port in communication with said second chamber.
